(19) United States
(12) Patent Application Publication
(10) Pub. No.: US 2015/0257713 A1
(43) Pub. Date: Sep. 17, 2015

(54) HEALTH MANAGEMENT SYSTEM, TERMINAL DEVICE, DISPLAY METHOD, AND CONTROL PROGRAM

(71) Applicant: PANASONIC INTELLECTUAL PROPERTY MANAGEMENT CO., LTD., Osaka (JP)

(72) Inventor: Shingo TSUKIHARA, Osaka (JP)

(21) Appl. No.: 14/639,092

(22) Filed: Mar. 4, 2015

Related U.S. Application Data
(63) Continuation of application No. PCT/JP2014/003642, filed on Jul. 9, 2014.

(30) Foreign Application Priority Data
Mar. 17, 2014 (JP) 2014-053057

(51) Int. Cl. A61B 5/00; G06F 3/0484; G06T 7/00

(52) U.S. Cl. CPC A61B 5/743; G06T 7/0012; G06F 3/04842; A61B 2560/0475

(57) ABSTRACT

A health management system for displaying, on a screen, information based on vital data obtained from a subject includes: a vital data storage unit storing vital data history; a priority level information storage unit storing information specifying a priority level for each of additional information items; a receiving unit receiving an operation of defining a first region and a second region in the screen; a selecting unit selecting an additional information item to be displayed, based on the priority level information and a size of each of the first and second region; and a display unit displaying (i) the vital data history obtained for a predetermined period and (ii) on a position related to the second region when the operation is received, additional information of the selected additional information item which is related to the vital data history obtained for the period corresponding to the first region.

### CROSS REFERENCE TO RELATED APPLICATIONS

This is a continuation application of PCT International Application No. PCT/JP2014/003642 filed on Jul. 9, 2014, designating the United States of America, which is based on and claims priority of Japanese Patent Application No. 2014-053057 filed on Mar. 17, 2014. The entire disclosures of the above-identified applications, including the specifications, drawings and claims are incorporated herein by reference in their entirety.

### FIELD

The present disclosure relates to a health management system for assisting health management, and to a technique of displaying, on a terminal device, information on measured vital data.

### BACKGROUND

Patent Literature (PTL) 1 discloses a technique of displaying a long-term trend graph, and displaying, within a specified frame, an enlarged graph resulting from enlarging the long-term trend graph and an average value of the data.

### CITATION LIST

### Patent Literature

[PTL 1] Japanese Unexamined Patent Application Publication No. 2010-049533

### SUMMARY

### Technical Problem

The present disclosure provides a health management system for displaying additional information of a suitable amount on a part of a screen on which the vital data history is displayed, and a technique related to the system, for allowing a viewer to view the vital data history on the screen with greater convenience than before.

### Solution to Problem

A health management system according to the present disclosure is a health management system for displaying, on a screen, information based on vital data successively obtained from a predetermined subject, the health management system including: a vital data storage unit configured to store vital data history in which the vital data is associated with a time at which the vital data is obtained; a priority level information storage unit configured to store priority level information specifying a priority level for each of a plurality of additional information items; a receiving unit configured to receive an operation of defining a first region and a second region in the screen; a selecting unit configured to select an additional information item to be displayed, based on the priority level information and a size of each of the first region and the second region for which the operation of defining is received by the receiving unit; and a display unit configured to (i) display, on the screen, the vital data history obtained for a predetermined period and (ii) display, on a position related to the second region when the operation of defining is received by the receiving unit, additional information of the additional information item which is selected by the selecting unit and is related to the vital data history obtained for a part of the predetermined period corresponding to the first region.

In addition, a terminal device according to the present disclosure is a terminal device for displaying, on a screen, information based on vital data successively obtained from a subject, the terminal device comprising: a vital data storage unit configured to store vital data history in which the vital data is associated with a time at which the vital data is obtained; a priority level information storage unit configured to store priority level information specifying a priority level for each of a plurality of additional information items; a receiving unit configured to receive an operation of defining a first region and a second region in the screen; a selecting unit configured to select an additional information item to be displayed, based on the priority level information and a size of each of the first region and the second region for which the operation of defining is received by the receiving unit; and a display unit configured to (i) display, on the screen, the vital data history obtained for a predetermined period and (ii) display, on a position related to the second region when the operation of defining is received by the receiving unit, additional information of the additional information item which is selected by the selecting unit and is related to the vital data history obtained for a part of the predetermined period corresponding to the first region.

In addition, a display method according to the present disclosure is a display method for displaying, on a screen of a terminal device, information based on vital data successively obtained from a subject, the display method comprising: storing vital data history in which the vital data is associated with a time at which the vital data is obtained; displaying the vital data history obtained for a predetermined period on the screen; obtaining priority level information specifying a priority level for each of a plurality of additional information items; receiving an operation of defining a first region and a second region in the screen; selecting an additional information item to be displayed, based on the priority level information and a size of each of the first region and the second region for which the operation of defining is received; and displaying, on a position related to the second region, additional information of the additional information item which is selected and is related to the vital data history obtained for a part of the predetermined period corresponding to the first region.

In addition, a control program according to the present disclosure is a control program for causing the terminal device to execute display processing of displaying information on a screen, based on vital data successively obtained from a subject, the display processing including: storing vital data history in which the vital data is associated with a time at which the vital data is obtained;

displaying the vital data history obtained for a predetermined period on the screen; obtaining priority level information specifying a priority level for each of a plurality of additional information items;

receiving an operation of defining a first region and a second region in the screen; selecting an additional information item to be displayed, based on the priority level information and a size of each of the first region and the second region for which the operation of defining is received; and displaying, on a position related to the second region, additional information of the additional information item which is selected and is related to the vital data history obtained for a part of the predetermined period corresponding to the first region.

### Advantageous Effects

A health management system, a terminal device, and the like according to the present disclosure selects an additional information item to be displayed on a screen, based on a size of a defined region, making it possible to display additional information of a suitable amount.

### BRIEF DESCRIPTION OF DRAWINGS

These and other objects, advantages and features of the disclosure will become apparent from the following description thereof taken in conjunction with the accompanying drawings that illustrate a specific embodiment of the present disclosure.

FIG. 1 is an outline of a configuration diagram of a health management system according to Embodiment 1.

FIG. 2 is a diagram illustrating a main hardware configuration of each of the devices and the like of the health management system.

FIG. 3 is a diagram illustrating an example of priority level information.

FIG. 4 is a diagram illustrating an example of the configuration and the details of a ratio table.

FIG. 5 is a functional block diagram of the health management system.

FIG. 6 is a flowchart illustrating display processing of vital data history.

FIG. 7 is a diagram illustrating an example of the screen image immediately after the operation of defining a region in a screen and starting the operation of enlargement.

FIG. 8 is a diagram illustrating an example of the screen image after the operation of defining a region in a screen and the operation of enlarging the region 1.8 times.

FIG. 9 is a flowchart illustrating an operation of an instructor terminal.

FIG. 10 is a functional block diagram of the health management system according to another embodiment.

FIG. 11 is a functional block diagram of the terminal device in the health management system according to another embodiment.

### DESCRIPTION OF EMBODIMENTS

In order to display additional information of a suitable amount related to a part of vital data history in response to an specifying operation of a viewer (subject) when the subject for health management views the vital data history on a screen of a terminal device, a health management system according to the present disclosure is a health management system for displaying, on a screen, information based on vital data successively obtained from a predetermined subject, the health management system including: a vital data storage unit configured to store vital data history in which the vital data is associated with a time at which the vital data is obtained; a priority level information storage unit configured to store priority level information specifying a priority level for each of a plurality of additional information items; a receiving unit configured to receive an operation of defining a first region and a second region in the screen; a selecting unit configured to select an additional information item to be displayed, based on the priority level information and a size of each of the first region and the second region for which the operation of defining is received by the receiving unit; and a display unit configured to (i) display, on the screen, the vital data history obtained for a predetermined period and (ii) display, on a position related to the second region when the operation of defining is received by the receiving unit, additional information of the additional information item which is selected by the selecting unit and is related to the vital data history obtained for a part of the predetermined period corresponding to the first region. Here, the vital data history is information including one or more combination of a measured value and a time and date of measurement of vital data. In addition, the vital data is information including a measured value of an item which can possibly be index of a state of health, such as a blood pressure, a blood glucose level, the number of steps, or a body weight, measured for a subject. Furthermore, the additional information item is information indicating types (items) of additional information that is information related to the vital data history. The position related to the second region is not limited to a position within the second region, and may be, for example, a position in a neighboring region of the second region. It is to be noted that the selection of the additional information item may be performed, for example, based on a ratio of the second region to the first region (dimensional ratio, area ratio, etc.), based on a difference in a size between the first region and the second region (difference in size, difference in area, etc.), or based on a ratio and a size of the second region. In addition, obtainment related to the vital data includes, for example, receiving an input of the subject's operation, receiving from a measuring device which measures the vital data of the subject. It is to be noted that the term “time” is a broader concept of all of the terms related to time such as year, month, day, and time of day.

According to the health management system, an additional information item to be displayed is selected based on the size of a region that is defined, and thus it is possible to display the additional information of a suitable amount in accordance with the defining of the region.

Here, for example, the selecting unit may select the additional information item to be displayed, based on the priority level information according to one of ratios that is a proportion of the size of the second region to the size of the first region, and the priority level information is information indicating an association between the additional information items and the ratios.

With this, when the subject enlarges the first region to define the second region, for example, the health management system is capable of displaying, on the screen, additional information of a suitable amount according to the ratio (enlargement rate).

In addition, for example, when the operation of defining is received by the receiving unit, the display unit may display the additional information in the second region, and the selecting unit may select more additional information items as the one of ratios increases.

With this, the additional information is displayed in the second region defined by the subject, and the defining of the region allows the subject to check the additional information of an amount according to the size of the region in the second region in addition to simultaneously checking the vital data history obtained for a predetermined period.

In addition, the display unit may determine a display form for the additional information according to the size of the second region, and display the additional information in the determined display form.

With this, the health management system is capable of displaying additional information properly in a form according to the size of the second region defined by the subject.

In addition, the selecting unit may select the additional information item to be displayed, based on the priority level information according also to the size of the second region.

With this, the health management system is capable of displaying additional information of a suitable amount for display in the second region defined by the subject.

In addition, the display unit may (i) display the vital data history obtained for the predetermined period in a graph form, and (ii) when the operation of defining is received by the receiving unit, enlarge and display in the second region the vital data history in the graph form at the one of ratios that is the proportion.

With this, the subject is allowed to easily check an overall tendency of the vital data history obtained for a predetermined period, and the defining of the region enables enlargement of a part of the graph for the checking.

In addition, the health management system may further include an analysis unit configured to calculate analysis information of the additional information item selected by the selecting unit, based on the vital data history obtained for a part corresponding to the first region, wherein the additional information displayed by the display unit includes the analysis information calculated by the analysis unit.

With this, the subject is allowed to check the analysis information of the vital data history on the screen.

In addition, the health management system may include a management apparatus which manages the vital data history and a subject terminal which obtains the vital data from the subject, the management apparatus and the subject terminal being capable of communicating with each other, wherein the subject terminal includes the screen, the receiving unit, and the display unit.

With this, the subject is allowed to view information on the vital data history on the screen of the subject terminal.

In addition, the management apparatus may be capable of communicating with an instructor terminal used for inputting instruction information for the subject by an instructor who has referred to the vital data history and include an instruction information storage unit configured to store the instruction information obtained from the instructor terminal, and the additional information displayed by the display unit may include the instruction information stored in the instruction information storage unit.

With this, the subject is allowed to check, on the screen, the instruction information provided by the instructor via the instructor terminal.

In addition, the priority level information stored in the priority level information storage unit may be updatable by the instructor terminal.

With this, the instructor is allowed to specify an order of priority of the additional information items displayed to the subject.

In addition, a terminal device according to the present disclosure is a terminal device for displaying, on a screen, information based on vital data successively obtained from a subject, the terminal device comprising: a vital data storage unit configured to store vital data history in which the vital data is associated with a time at which the vital data is obtained; a priority level information storage unit configured to store priority level information specifying a priority level for each of a plurality of additional information items; a receiving unit configured to receive an operation of defining a first region and a second region in the screen; a selecting unit configured to select an additional information item to be displayed, based on the priority level information and a size of each of the first region and the second region for which the operation of defining is received by the receiving unit; and a display unit configured to (i) display, on the screen, the vital data history obtained for a predetermined period and (ii) display, on a position related to the second region when the operation of defining is received by the receiving unit, additional information of the additional information item which is selected by the selecting unit and is related to the vital data history obtained for a part of the predetermined period corresponding to the first region.

In addition, a display method according to the present disclosure is a display method for displaying, on a screen of a terminal device, information based on vital data successively obtained from a subject, the display method comprising: storing vital data history in which the vital data is associated with a time at which the vital data is obtained; displaying the vital data history obtained for a predetermined period on the screen; obtaining priority level information specifying a priority level for each of a plurality of additional information items; receiving an operation of defining a first region and a second region in the screen; selecting an additional information item to be displayed, based on the priority level information and a size of each of the first region and the second region for which the operation of defining is received; and displaying, on a position related to the second region, additional information of the additional information item which is selected and is related to the vital data history obtained for a part of the predetermined period corresponding to the first region.

In addition, a control program for causing a terminal device to perform display processing is a control program for causing the terminal device to execute display processing of displaying information on a screen, based on vital data successively obtained from a subject, the display processing including: storing vital data history in which the vital data is associated with a time at which the vital data is obtained;

displaying the vital data history obtained for a predetermined period on the screen; obtaining priority level information specifying a priority level for each of a plurality of additional information items; receiving an operation of defining a first region and a second region in the screen; selecting an additional information item to be displayed, based on the priority level information and a size of each of the first region and the second region for which the operation of defining is received; and displaying, on a position related to the second region, additional information of the additional information item which is selected and is related to the vital data history obtained for a part of the predetermined period corresponding to the first region.

According to the above, an additional information item to be displayed on a screen of the terminal device is selected based on the size of a region that is defined by the subject, and thus it is possible to display the additional information of a suitable amount which corresponds to the defining of the region.

These general and specific embodiments include one or more combinations of an apparatus, a system, a method, an integrated circuit, a computer program, and a computer-readable recording medium.

Hereinafter, non-limiting embodiments are described in greater detail with reference to the accompanying Drawings. However, description that is too detailed will be omitted in some cases. For example, detailed description for an already well-known item or redundant description for a substantially equal configuration will be omitted in some cases. This is for the purpose of preventing the following description from being unnecessarily redundant and facilitating understanding of those skilled in the art.

It is to be noted that the inventors provide the Drawings and the following description for supporting those skilled in the art to fully understand the present disclosure, and thus do not intend that the Drawings and the following description will limit the subject matter of Claims. In other words, the numerical values, shapes, materials, structural elements, the arrangement and connection of the structural elements, steps, the processing order of the steps, etc. shown in the following embodiments are mere examples, and therefore do not limit the scope of the appended Claims and their equivalents. Among the structural elements in the following embodiments, structural elements not recited in any one of the independent claims are described as arbitrary structural elements. In addition, each diagram is a schematic diagram and not necessarily strictly illustrated.

### Embodiment 1

In Embodiment 1, a health management system 1 will be described which assists health management of a predetermined subject by mainly displaying information and the like based on vital data obtained from the subject.

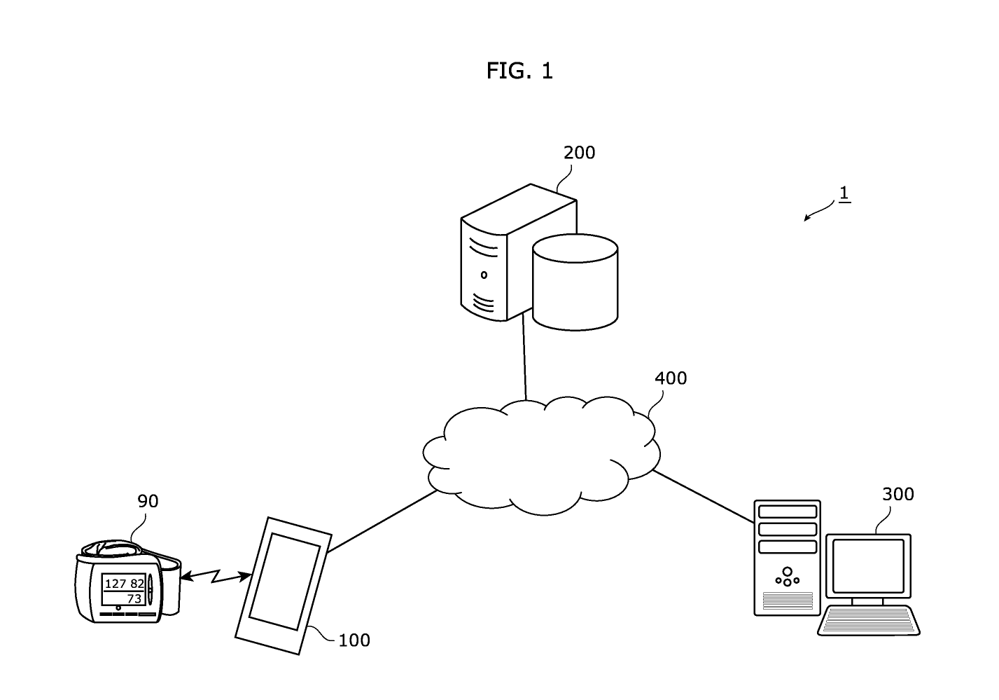

Hereafter, Embodiment 1 will be described with reference to FIG. 1 to FIG. 9.

[1-1 Configuration]

FIG. 1 is an outline of a configuration diagram of the health management system 1 according to Embodiment 1.

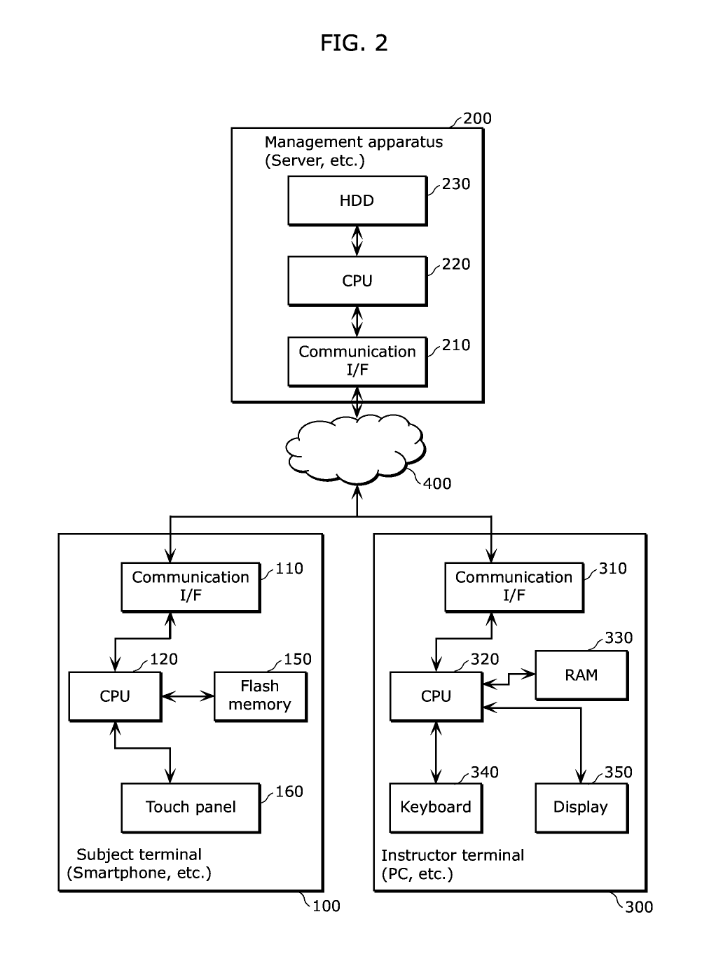

As illustrated in the diagram, the health management system 1 includes: a subject terminal 100; a management apparatus 200; an instructor terminal 300; and an Internet network 400 that is a network connecting these devices such that these devices can communicate with each other. An example of a principal hardware configuration of each of the devices is illustrated in FIG. 2.

The subject terminal 100 is a terminal device such as a smartphone and a tablet which are operated by a subject, and includes: a memory (a flash memory 150 etc.); a processor (CPU 120); an input device and a display device (touch panel 160); and a communication interface (I/F) 110, and the like. The communication I/F 110 is a communication circuit or the like which is configured to exchange information with other devices via the Internet network 400 and other communication tools. The memory is a ROM or a RAM, for example. In the memory, a control program for implementing processing of each functional unit described below, setting values for use in the control program, and the like are prestored in the memory, and the memory is also used for temporarily storing each value used in executing the control program by the processor. The flash memory 150 temporarily stores, for example, calculation results calculated by the CPU 120, or information obtained via the communication I/F 110. The subject terminal 100 is capable of communicating with a measuring device which measures vital data of the subject using a radio communication technique such as Bluetooth (registered trademark). It is to be noted that the vital data is, for example, so-called biological information such as a blood pressure and a blood glucose level, information on an amount of activity such as the number of steps, information according to a physical feature such as a body weight.

The management apparatus 200 is a server including: a storage medium having relatively large capacity such as a memory, a processor, (a CPU 220), and a hard disk device (HDD) 230; a communication I/F 210; and the like. The communication I/F 210 is a communication circuit or the like which is configured to exchange information with other devices via the Internet network 400 and other communication tools. The HDD 230 stores information obtained via the communication I/F 210, for example.

The instructor terminal 300 is a terminal device such as a personal computer (PC) which is operated by a person (an instructor) who remotely provides a subject with instructions on health, and includes: a memory (a RAM 330 etc.), a processor (a CPU 320), an input device (a keyboard 340 etc.), a display 350, and a communication I/F 310, and the like. The communication I/F 310 is a communication circuit or the like which is configured to exchange information with other devices via the Internet network 400 and other communication tools. The RAM 330 temporarily stores information obtained via the communication I/F 310, for example.

In the above and following description, it is assumed that the subject terminal 100 is a smartphone carried by the subject, the management apparatus 200 is a server, and the instructor terminal 300 is a PC.

The following describes an example of usage of the health management system 1.

First, vital data measured for the subject and information on the time and date of the measurement are received by the subject terminal 100 by, for example, input by the subject via the touch panel 160 or transmission from the measuring device 90. Then, the vital data and the like are transmitted to the management apparatus 200 from the subject terminal 100, and accumulated (registered) as vital data history on, for example, the HDD 230. For example, the vital data history is managed by a database. The vital data includes a measured value of an item which can possibly be index of a state of health, such as a blood pressure, a blood glucose level, the number of steps, or a body weight, measured by, for example, the measuring device 90 or the like. The vital data history accumulated in the management apparatus 200 includes a measured value of vital data and time and date of the measurement, and may further include information such as information for identifying the subject (a subject label), information for identifying an item of the vital data (a vital item label), and the like. For example, when a subject A inputs a body weight of 60.0 Kg measured at 10:00 on Feb. 17, 2014, data (data record) that “subject label: A, vital item label: body weight, measured value: 60.0, time and date of the measurement: 10:00 on Feb. 17, 2014” is added to the management apparatus 200 as information included in the vital data history.

The instructor downloads and views the vital data and the like of the subject from the management apparatus 200 onto the instructor terminal 300. The instructor provides each subject with instructions on health for preventing diseases, for example, through an interview if necessary, a telephone call, and the like (for example, through a video call via the Internet network 400, transmission of instruction information via the management apparatus 200, etc.). Then, the details of the instructions on health are inputted to the instructor terminal 300 and uploaded and registered on the management apparatus 200 as the instruction information. The health management system 1, although only a single subject terminal is illustrated in FIG. 1 etc., may include a subject terminal for each subject, and thus it is possible, for example, to implement an operation that a single instructor instructs approximately 20 to 30 subjects. It is to be noted that the instruction information is information including a comment for preventing diseases or maintaining health provided as an advice from the instructor with reference to the vital data, a target value of the vital data, and the like. The instruction information is recorded (registered) by the HDD 230, for example, of the management apparatus 200 in association with any items of data record (vital data etc.) which has been referred to by the instructor in the vital data history. In addition, the instructor, through uploading, can record (register) the instruction information on the management apparatus 200 and record (register) priority level information on each subject on the management apparatus 200.

Then, when the subject operates the subject terminal 100 for viewing the vital data history (the measurement results obtained up to the present), vital data history obtained for a predetermined period which has been downloaded from the management apparatus 200 is displayed in a graph form on the screen (main screen) of the subject terminal 100. The subject operates the subject terminal 100 to display additional information and an enlarged graph of a part of the vital data history on a part of region (sub screen) of the screen (main screen) of the subject terminal 100, and views the displayed images. Types (items) of the additional information (analysis of the vital data such as an average value, instruction information inputted by the instructor, and the like) displayed on the sub screen is determined based on the priority level information.

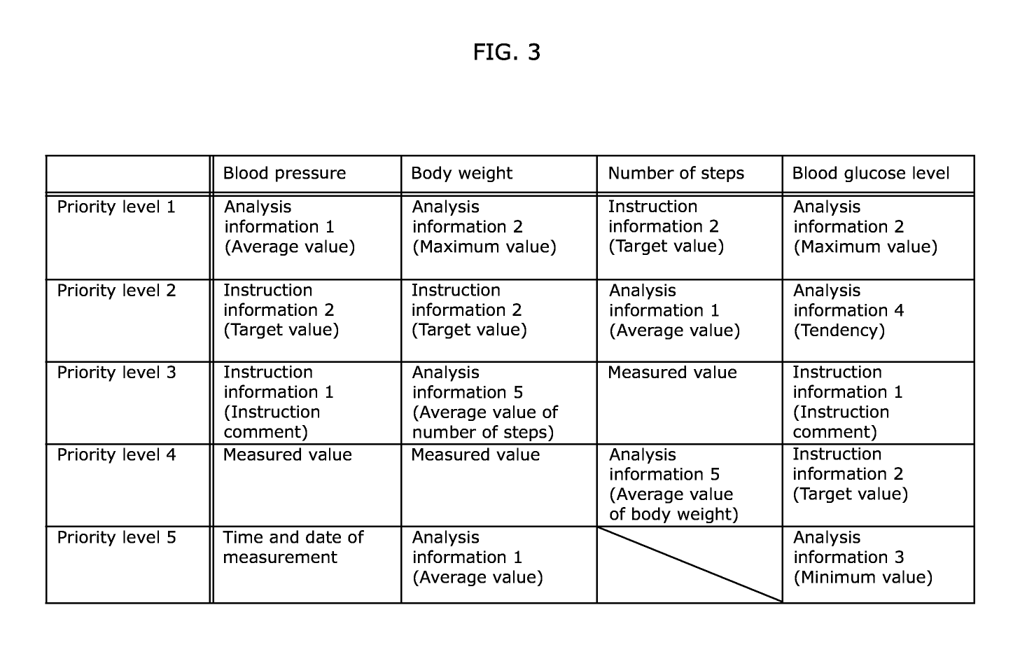

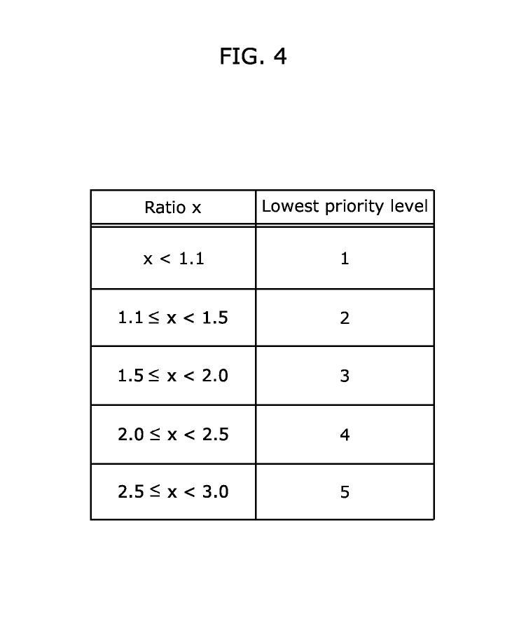

The priority level information is information indicating a priority level of each of the items of additional information that can be displayed on the sub screen. A table which is an example of the priority level information is illustrated in FIG. 3. As illustrated in the diagram, as items of the additional information (additional information items) on a blood pressure, which can be displayed on the sub screen, include: analysis information 1 (an average value); instruction information 1 (instruction comment), an instruction information 2 (a target value), a measured value and time and date of the measurement, in descending order of priority. Upon operation by the subject to define and enlarge a part of region in the screen, in other words, when the subject performs operation of defining and enlarging a part of the graph (a part of the period), which is displayed on the screen, of the vital data history obtained for a predetermined period, up to which of the priority levels the additional information is to be displayed is determined according to the ratio (enlargement rate). As the ratio is smaller, the number of the additional information items to be displayed on the sub screen decreases; in other words, only the additional information items with high priority levels are displayed. As the ratio is larger, additional information items even with low priority levels are more likely to be displayed. Based on the example of the priority level information illustrated in FIG. 3, for example, in the case where a part of a graph of the blood pressure is defined and then enlarged, (i) analysis information 1 (average value) is displayed together with the enlarged graph when the ratio is small, (ii) the analysis information 1 (average value) and instruction information 2 (target value) are displayed together with the enlarged graph when the ratio increases a little, and (iii) the analysis information 1 (average value), the instruction information 2 (target value), and instruction information 1 (instruction comment) are displayed together with the enlarged graph when the ratio further increases. When the ratio increases to the maximum, the analysis information 1 (average value), the instruction information 2 (target value), the instruction information 1 (instruction comment), and a measured value and time and date of the measurement are displayed together with the enlarged graph. Since the measured value and the time and date of the measurement have values for each time and date of measurement during a specified period, a plurality of measured values and the times and dates of the measurements may be displayed. Up to which of the priority levels information is to be displayed with respect to a ratio is associated previously using a table or a calculating formula, for example. FIG. 4 illustrates an example of the table (a ratio table). In FIG. 4, the lowest priority level indicates up to which of the priority levels information is to be displayed. If the lowest priority level is 3, it indicates that additional information items having the priority levels 1 to 3 are to be displayed. In the example of FIG. 4, when the ratio is 2, for example, the lowest priority level is 4, and thus the additional information items having the priority levels 1 to 4 are displayed. The ratio table is structured such that the larger the ratio is, the larger the amount of the additional information items is. In addition, the analysis information is information calculated by, for example, the CPU 120 in the subject terminal 100, based on the vital data history downloaded from the management apparatus 200. The analysis information is, for example, an average value in a specified period (analysis information 1), a maximum value (analysis information 2), a minimum value (analysis information 3), a tendency (analysis information 4), an average value of another vital data history having a high correlation (analysis information 5), or the like. Here, a tendency is an index indicating, for example, whether a measured value of the vital data is on the rise or on the decrease in a specified period. In addition, another vital data history having a high correlation is data of the number of steps with respect to the body weight, for example. For example, the subject terminal 100 displays, on a sub screen, an average value of the number of steps during a specified period based on the additional information item of the priority level 3, when a subject enlarges the graph of the body weight with a sufficient ratio. It is to be noted that the additional information is information related to the vital data history, such as analysis information, instruction information, and a measured value, as is obvious from the items (additional information items) exemplified in FIG. 3.

The following describes in detail each of the devices and the like which are included in the above-described health management system 1.

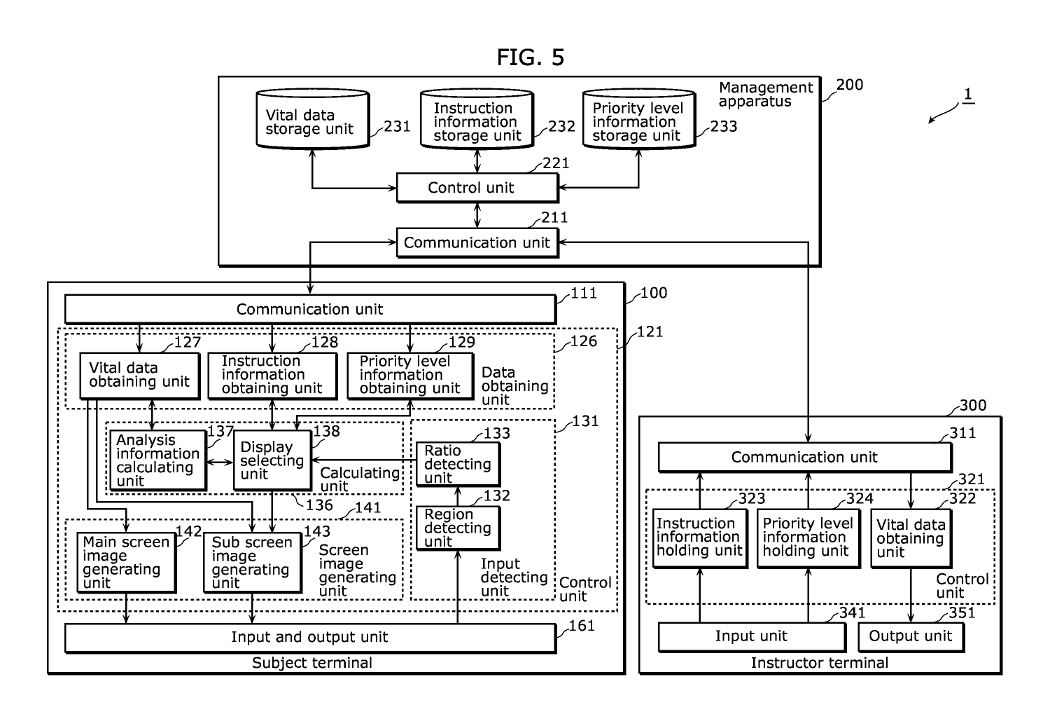

FIG. 5 is a functional block diagram of the health management system 1.

[1-1-1. Functional Configuration of the Subject Terminal]

The subject terminal 100 functionally includes a communication unit 111, a control unit 121, and an input and output unit 161, as illustrated in FIG. 5. The following describes each of the functional units (functional structural elements).

The communication unit 111 has a function of obtaining and transmitting to the control unit 121, vital data, instruction information, and priority level information registered on the management apparatus 200, and transmitting (uploading) vital data history to the management apparatus 200 based on the vital data provided successively. When the vital data is transmitted from the measuring device 90, the communication unit 111 may have a function of receiving and obtaining the vital data, and transmitting vital data history to the management apparatus 200 based on the vital data. The communication unit 111 is implemented by the communication I/F 110 which operates under the control of the processor (CPU 120) which executes a control program stored in a memory. It is to be noted that the communication unit 111 may transmit vital data history every time the vital data is obtained, or may collectively transmit a predetermined amount of the vital data history.

The control unit 121 has a function of performing various controls and calculations in the subject terminal 100, and includes a data obtaining unit 126, an input detecting unit 131, a calculating unit 136, and a screen image generating unit 141. The communication unit 121 is implemented by a processor (CPU 120) and a memory (flash memory 150 etc.) which executes a control program stored in a memory. Here, the data obtaining unit 126 has a function of obtaining data via the communication unit 111, and includes a vital data obtaining unit 127, an instruction information obtaining unit 128, and a priority level information obtaining unit 129. The input detecting unit 131 has a function of detecting an operation conducted by the subject, and includes a region detecting unit 132 and a ratio detecting unit 133. The calculating unit 136 has a function of selecting information to be output according to the operation conducted by the subject and performing calculation, and includes an analysis information calculating unit 137 and a display selecting unit 138. The screen image generating unit 141 has a function of generating a screen image (a main screen image and a sub screen image) to be output to the display device (the touch panel 160), and includes a main screen image generating unit 142 and a sub screen image generating unit 143.

The vital data obtaining unit 127 has a function of obtaining vital data history from the management apparatus 200 via the communication unit 111, storing the obtained vital data history onto a part of the region of a memory such as the flash memory 150, and transmitting the vital data history to the analysis information calculating unit 137 and the screen image generating unit 141.

The instruction information obtaining unit 128 has a function of obtaining instruction information from the management apparatus 200 via the communication unit 111, storing the obtained instruction information onto a part of the region of a memory such as the flash memory 150, and transmitting the instruction information to the display selecting unit 138.

The priority level information obtaining unit 129 has a function of obtaining priority level information from the management apparatus 200 via the communication unit 111, storing the obtained priority level information onto a part of the region of a memory such as the flash memory 150, and transmitting the priority level information to the display selecting unit 138.

The input and output unit 161 has an output function of displaying a main screen image etc. (for example, a graph of the vital data history obtained for a predetermined period) generated by the screen image generating unit 141 and an input function of receiving an operation conducted by a subject, such as defining of a region for the graph of the vital data history (tap), enlargement (pinch out) or reduction (pinch in), and the like. This input and output unit 161 is implemented by an input device and a display device (for example, the touch panel 160).

The region detecting unit 132 has a function of detecting a region which is a target for enlargement or reduction and a period, according to the operation of defining a part of the region of the graph displayed on the screen, which has been received by the input and output unit 161. More specifically, when the subject defines, with a tapping using two fingers, a region corresponding to one week of a graph for one month on the main screen image outputted from the input and output unit 161, the region detecting unit 132 detects positions of tapped two points and detects a range (size) of a region enclosed by the two points and a specified period (one week) corresponding to the region between the two points. The region detecting unit 132 has another function of detecting a region resulting from the enlargement or reduction. More specifically, when the subject performs the operation of pinch out (to expand a gap between two fingers) or pinch in (to reduce a gap enclosed by two fingers), the region detecting unit 132 detects the positions of two fingers on the input and output unit 161 and detects a range (size) of the region between two fingers after the operation (pinch out or pinch in). It is to be noted that the range of the region after the operation is the sub screen image, and an enlarged (reduced) graph and additional information are to be displayed in the sub screen image.

The ratio detecting unit 133 has a function of calculating and thereby detecting a ratio (dimensional ratio) that is a ratio of the range (size) of the region which is a target for enlargement or reduction and detected by the region detecting unit 132 to the range (size) of the region resulting from the enlargement or reduction. More specifically, when the subject performs the operation of pinch out (expanding a gap between two fingers) or pinch in (reducing a gap between two fingers), the ratio detecting unit 133 detects a dimensional ratio before and after the operation (pinch out or pinch in). Information on the specified period detected by the region detecting unit 132 and the information on the ratio detected by the ratio detecting unit 133 are transmitted to the display selecting unit 138.

The analysis information calculating unit 137 calculates analysis information based on the instruction related to the specified period and transmitted from the display selecting unit 138 and the vital data history obtained by the vital data obtaining unit 127. Examples of the analysis information include: an average value in a specified period (i.e. an average value of the measured values measured during the specified period in the vital data history), a maximum value in the specified period, a minimum value in the specified period, a tendency of the specified period (an upward trend, a downward trend, flat, etc.), an average value in a specified period of another vital data having a high correlation, and the like.

The display selecting unit 138 has a function of selecting an item of additional information (additional information item) to be displayed together with a graph enlarged in the sub screen image, by referring to (i) the ratio which is related to enlargement (reduction) of the region and obtained from the ratio detecting unit 133 and (ii) the priority level information obtained from the priority level information obtaining unit 129. In addition, the display selecting unit 138 has another function of transmitting the additional information obtained from the vital data obtaining unit 127, the instruction information obtaining unit 128, or the analysis information calculating unit 137 to the sub screen image generating unit 143, by instructing the vital data obtaining unit 127, the instruction information obtaining unit 128, or the analysis information calculating unit 137 to output the selected additional information.

The main screen image generating unit 142 has a function of generating a main screen image including the vital data history represented in a graph form to be displayed on the input and output unit 161 based on the vital data history obtained from the vital data obtaining unit 127. To be represented in a graph form means that, for example, the vital data history is represented in a graph in which the times and dates of the measurement of the vital data (measured values) are represented in the horizontal axis and the measured values are represented in the vertical axis. The main screen image has content to be displayed over the whole screen of the display device (touch panel 160), for example.

The sub screen image generating unit 143 has a function of generating a sub screen image to be superimposed and displayed on a part of region of the main screen image according to an operation of pinch out or pinch in performed by the subject while the main screen image is displayed. The sub screen image corresponds to the region which has been enlarged (reduced) resulting from the operation such as pinch out performed by the subject. Additional information obtained from the display selecting unit 138 together with the enlarged or reduced graph is displayed on the sub screen image.

[1-1-2. Functional Configuration of the Management Apparatus]

The management apparatus 200 functionally includes a communication unit 211, a control unit 221, a vital data storage unit 231; an instruction information storage unit 232; and a priority level information storage unit 233, as illustrated in FIG. 5 The following describes each of the functional structural elements.

The communication unit 211 has a function of communicating with the subject terminal 100 and the instructor terminal 300 and is implemented by the communication I/F 210 which operates under the control of the processor (CPU 220) which executes a control program stored in a memory.

The control unit 221 has a function of performing various controls in the management apparatus 200, and is implemented by the processor (CPU 220) which executes the control program stored in a memory.

The vital data storage unit 231 is implemented by a part of the storage medium of the HDD 230 and has a function of storing (accumulating) the vital data history registered by the subject. The vital data storage unit 213 stores vital data history for each of one or more subjects, and for example, obtains a subject label upon obtaining the vital data history from each of the subject terminals to identify each of the subjects, thereby managing the vital data history for each of the subjects.

The instruction information storage unit 232 is implemented by a part of the storage medium of the HDD 230 and has a function of storing (accumulating) the instruction information registered by the instructor. The instruction information storage unit 232 stores the instruction information for each of the one or more subjects, and obtains a subject label when the instruction information is registered by the instructor terminal 300, thereby managing the instruction information for each of the subjects. It is to be noted that the instruction information may be managed in association with identification information for identifying the instructor who has registered the instruction information.

The priority level information storage unit 233 is implemented by a part of the storage medium of the HDD 230 and has a function of storing (accumulating) the priority level information previously determined or registered by the instructor. The priority level information storage unit 233 stores the priority level information for each of the one or more subjects. A plurality of basic patterns of the priority level information commonly available to a plurality of subjects may be prepared for each symptom such as a high blood pressure, likelihood of developing metabolic syndrome or diabetes, and any of the basic patterns may be set for each of the subjects as the priority level information. In addition, details of the priority level information may be set independently for each of the subjects. It is to be noted that the priority level information for each of the subjects is managed in association with the subject label for identifying the subject.

[1-1-3. Functional Configuration of the Instructor Terminal]

The instructor terminal 300 functionally includes a communication unit 311, a control unit 321, an input unit 341, and an output unit 351, as illustrated in FIG. 5. The following describes each of the functional structural elements.

The communication unit 311 has a function of communicating with the management apparatus 200. More specifically, the communication unit 311 has a function of obtaining and transmitting, to the control unit 321, the vital data history registered on the management apparatus 200, and transmitting the instruction information and the priority level information obtained from the control unit 321 to the management apparatus 200. The communication unit 311 is implemented by the communication I/F 310 which operates under the control of the processor (CPU 320) which executes a control program stored in a memory.

The communication unit 321 has a function of performing various controls in the instructor terminal 300, and is implemented by a processor (CPU 320) and a memory (RAM 330 etc.) which executes a control program stored in the memory. The control unit 321 includes a vital data obtaining unit 322, an instruction information holding unit 323, and a priority level information holding unit 324.

The vital data obtaining unit 322 has a function of obtaining the vital data history of a subject from the management apparatus 200 via the communication unit 311, and transmitting the obtained vital data history to the output unit 351.

The instruction information holding unit 323 has a function of obtaining and temporarily holding the instruction information inputted via the input unit 341 by the instructor (information related to a health instruction conducted based on the vital data history of the subject) and transmitting the instruction information to the management apparatus 200 via the communication unit 311. The transmission is carried out in response to an instruction issued by the instructor after checking for an error in the temporarily held instruction information, for example.

The priority level information holding unit 324 has a function of obtaining the priority level information from the input unit 341 when the instructor has changed the priority level information for the subject, temporarily holding the obtained priority level information, and transmitting to the management apparatus 200 via the communication unit 311. The transmission is carried out in response to an instruction issued by the instructor after checking for an error in the temporarily held priority level information, for example.

The input unit 341 is implemented by an input device (keyboard 340 etc.) and has a function of receiving an input operation performed by the instructor. The input unit 341 receives the instruction information and the priority level information provided by the instructor, and transmits the provided instruction information and the priority level information to the instruction information holding unit 323 and the priority level information holding unit 324.

The output unit 351 is implemented by the display 350 or the like, and has a function of displaying or outputting the vital data history etc. of the subject obtained by the vital data obtaining unit 322 via the communication unit 311 from the management apparatus 200.

[1-2 Operation]

The following describes an operation of the health management system 1 which includes the above-described configuration.

[1-2-1. Operation Related to the Subject Terminal]

The subject terminal 100 obtains vital data that is a measurement result of the subject, through an input performed by the subject or by receiving from the measuring device 90. Then, the subject terminal 100 transmits vital data history of the obtained vital data to the management apparatus 200. The vital data history is accumulated and managed by the management apparatus 200.

When a subject views measurement results (vital data history) obtained up to the present, the subject terminal executes display processing of the vital data history. Here, an example of displaying a result of measuring a blood pressure will be described with reference to the example of the priority level information in FIG. 3 and the ratio table in FIG. 4.

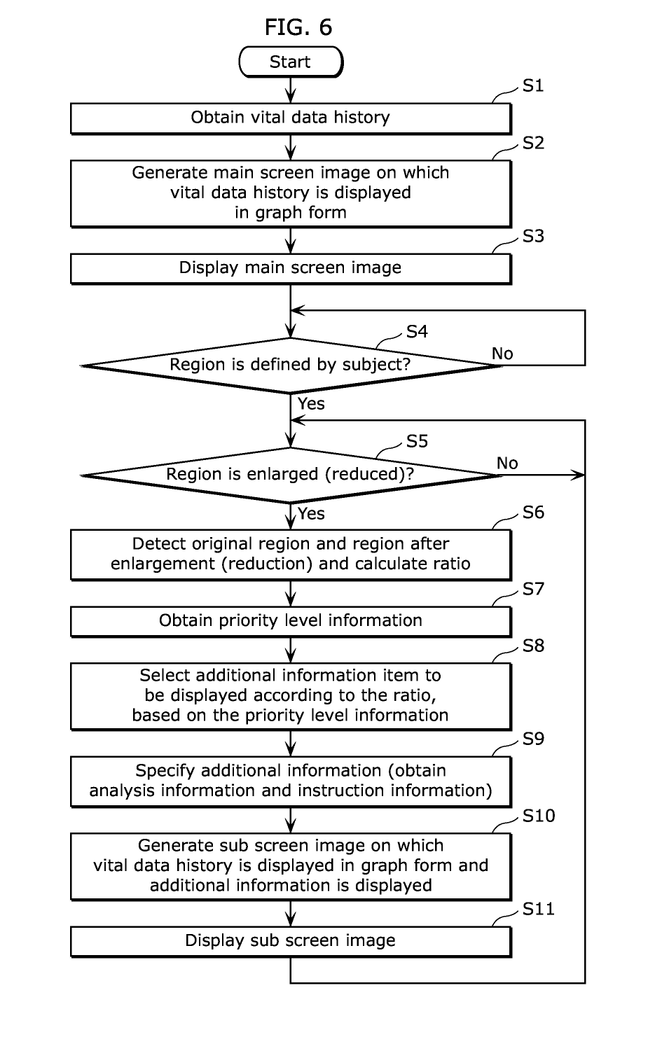

FIG. 6 is a flowchart illustrating display processing of vital data history. The following describes display processing of vital data history at the subject terminal 100 with reference to the flowchart illustrated in FIG. 6.

First, the subject terminal 100 obtains, through the communication unit 111 and the vital data obtaining unit 127, vital data history registered on the vital data storage unit 231 of the management apparatus 200 (Step S1). Next, the main screen image generating unit 142 obtains vital data history obtained for a predetermined period (this month, for example) from the vital data obtaining unit 127, represent the obtained vital data history in a graph form, and generates a main screen image including the graph (Step S2). It is to be noted that the predetermined period may be changed according to an operation by the subject. Subsequently, the subject terminal 100 displays the main screen image using the input and output unit 161 (Step S3). With this, a graph in which the times and dates of the measurement of the vital data (measured values of blood pressures) for the predetermined period are represented in the horizontal axis and the vital data is represented in the vertical axis is displayed on a screen such as the touch panel 160 of the subject terminal 100.

Then, when the subject who is viewing the graph of the vital data history defines a region in the main screen image on the touch panel 160 by tapping the region with two fingers, and thereby defining a part of the period related to the graph displayed on the region, the input and output unit 161 receives the definition (Step S4). Subsequently, upon an operation of enlarging (reducing) the region (Step S5), in other words, upon an operation of pinch out (enlargement) or pinch in (reduction) by the subject with two fingers, the input and output unit 161 receives the operation, and the subject terminal 100 moves to the next process in Step S6.

That is, the region detecting unit 132 detects (i) a specified period corresponding to the range of the region defined by two positions tapped by the subject and (ii) the range of the region after enlargement or reduction, and the ratio detecting unit 133 calculates a ratio (Step S6). Then, the priority level information obtaining unit 129 obtains priority level information from the management apparatus 200 (Step S7), and the display selecting unit 138 selects, based on the priority level information obtained from the priority level information obtaining unit 129, an additional information item to be displayed in the sub screen image according to the ratio calculated by the ratio detecting unit 133 (Step S8). It is to be noted that the obtaining of the priority level information performed in Step S7 may be performed once at the same time when obtaining the vital data history in Step S1.

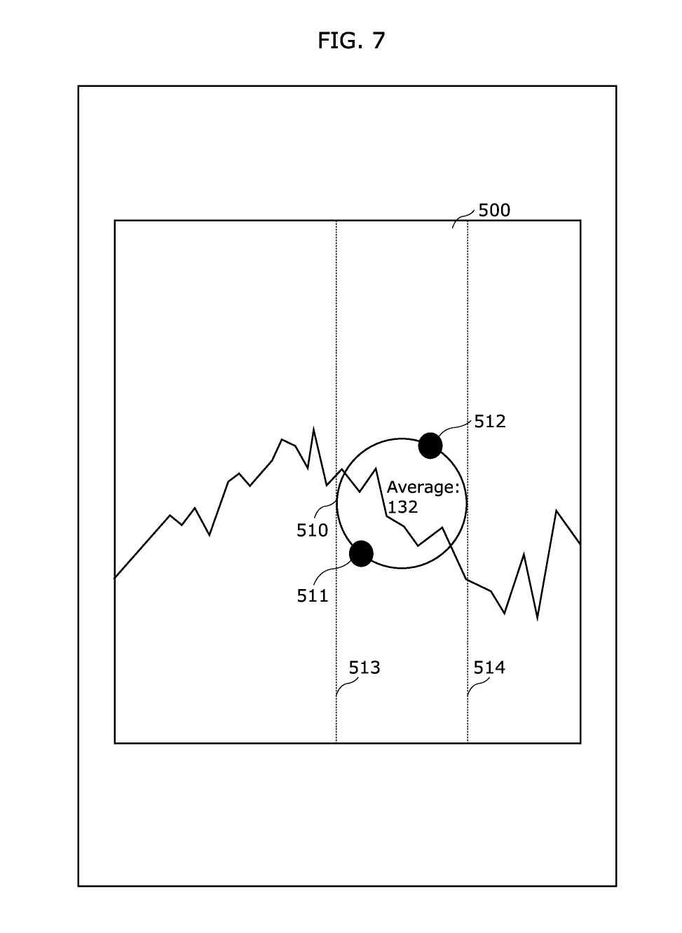

FIG. 7 illustrates an example of screen image immediately after starting the operation of pinch out (enlargement) following the defining of the region by the subject. In a main screen image 500 in the diagram, a position 511 and a position 512 each denote a position tapped by a corresponding one of the two fingers. The region detecting unit 132 calculates a defined region 510 (sub screen image) shaped in a circle with a diameter that extends between the position 511 and the position 512 which are tapped, and detects, as a specified period, a period corresponding to a gap between a perpendicular 513 and a perpendicular 514 that circumscribe the defined region 510 in the horizontal axis (measured times and dates) of the graph of blood pressure (for example, one week from February 11 to February 17). It is to be noted that the defined region 510 may be fitted into a grid having a unit of date in the horizontal axis, in order to specify a period by the unit of date. In addition, the specified period may be detected using calculation for rounding a position into the unit of date, without performing the fitting into a grid.

Since the ratio is approximately 1 in a state immediately after the operation of pinch out (enlargement) is started, the lowest priority level is 1 according to the example of the ratio table illustrated in FIG. 4, and the priority level 1 for the blood pressure indicates analysis information 1 (average value) according to the example of the priority level information illustrated in FIG. 3. Thus, the display selecting unit 138 selects the analysis information 1 (average value) as an additional information item to be displayed.

The display selecting unit 138, after selecting the additional information item, specifies additional information to be included in the sub screen image according to the selected additional information item (Step S9). In other words, when the additional information item indicates a measured value or a time and date of measurement, the display selecting unit 138 obtains and specifies the measured value or the time and date of measurement from the vital data history obtained via the vital data obtaining unit 127. In addition, when the additional information item indicates instruction information, the display selecting unit 138 obtains and specifies the instruction information via the instruction information obtaining unit 128. Furthermore, when the additional information item indicates analysis information, the display selecting unit 138 obtains and specifies the analysis information calculated by the analysis information calculating unit 137.

In the following description, it is assumed that the analysis information 1 (average value) is selected by the display selecting unit 138 as additional information to be displayed. The analysis information calculating unit 137 calculates, based on the vital data history in a specified period (for example, from February 11 to February 17), the analysis information 1 that is an average value thereof, and the display selecting unit 138 identifies the analysis information 1 as the additional information to be displayed (Step S9). It is to be noted that the analysis information calculating unit 137, based on the specified period, calculates at least one which is selected as additional information item to be displayed, from among the analysis information items (average value, maximum value, minimum value, tendency, etc.).

The sub screen image generating unit 143 enlarges and represents a graph of vital data history according to the ratio based on the vital data history in the specified period obtained from the vital data obtaining unit 127, and generates a sub screen image representing the analysis information 1 (average value) that is additional information obtained from the display selecting unit 138 (Step S10). With this, the sub screen image includes additional information and a graph of which the size is adjusted to the size of the sub screen image (enlarged defined region). The input and output unit 161 superimposes and displays, on the main screen image, the sub screen image generated by the sub screen image generating unit 143 (Step S11). As a result, the average value is displayed together with the graph in the defined region 510 (sub screen image) as illustrated in FIG. 7.

In addition, when the subject continues to pinch out (enlargement) with the two fingers used in the tapping (Step S5), the region detecting unit 132 and the ratio detecting unit 133 detect a range of the enlarged region and calculate a ratio (Step S6). The following description assumes that the defined region is enlarged 1.8 times as a result of the pinch out performed by the subject. The display selecting unit 138 selects an additional information item to be displayed according to the ratio (1.8 times) based on the priority level information obtained from the priority level information obtaining unit 129 (Step S8). According to the example of the ratio table illustrated in FIG. 4, since the lowest priority level corresponding to the ratio (1.8 times) is 3, additional information items having the priority levels 1 to 3 as to the blood pressure are selected. Thus, according to the example of the priority level information illustrated in FIG. 3, the display selecting unit 138 selects the analysis information 1 (average value) having the priority level 1, the instruction information 2 (target value) having the priority level 2, and the instruction information 1 (instruction comment) having the priority level 3. Subsequently, the analysis information calculating unit 137 calculates an average value based on the vital data history in a specified period (for example, from February 11 to February 17), and obtains a target value and the instruction comment of the instruction information corresponding to the specified period from the management apparatus 200 via the instruction information obtaining unit 128. It is to be noted that the instruction information obtaining unit 128 may obtain and hold instruction information once at the same time when obtaining the vital data history in Step S1, instead of obtaining the instruction information from the management apparatus 200 according to an instruction from the display selecting unit 138. The display selecting unit 138 transmits, to the sub screen image generating unit 143, the average value obtained from the analysis information calculating unit 137 and the instruction comment and the target value obtained from the instruction information obtaining unit 128.

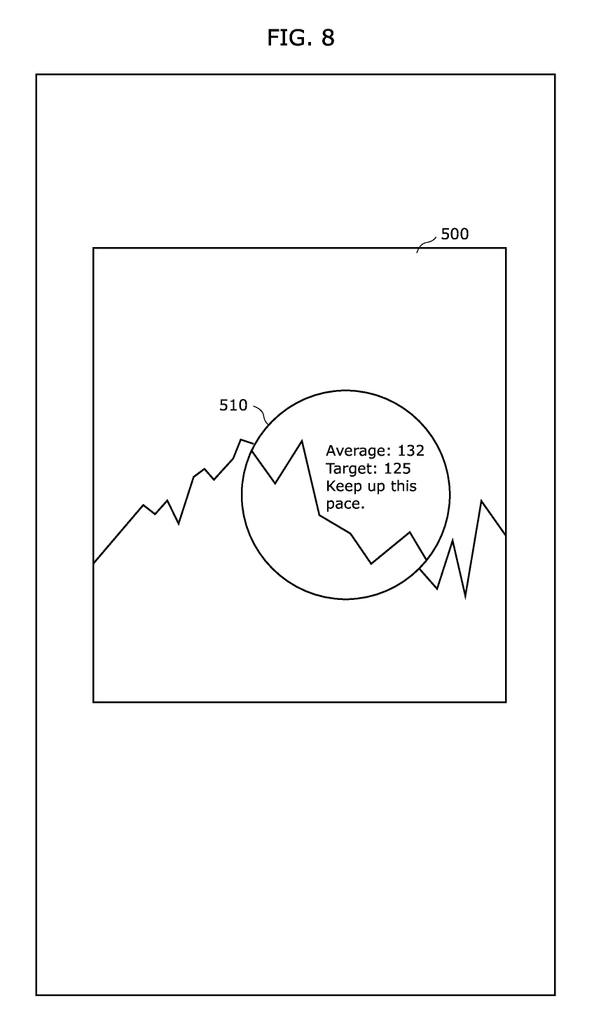

The sub screen image generating unit 143 generates a sub screen image indicating the analysis information 1 (average value), the instruction information 2 (target value), and the instruction information 1 (instruction comment) which are additional information items, while enlarging 1.8 times and display the graph of the vital data history (Step S10). The input and output unit 161 superimposes and displays the sub screen image onto the main screen image (Step S11). FIG. 8 illustrates an example of the screen image in this state. As illustrated in FIG. 8, in the defined region 510 (sub screen image) defined by the operation of pinch out by the subject, a part of the graph displayed in the main screen image 500 is enlarged 1.8 times and displayed, and the additional information items including an average value, a target value, and an instruction comment are displayed.

It is to be noted that, when the subject moves two fingers away from the touch panel 160 after tapping the touch panel 160 with the two fingers, the process may return to Step S4 in FIG. 6 and new tapping (defining a region) may be waited. It is to be noted that a sub screen image already displayed may be deleted when displaying a sub screen image in response to the new tapping.

According to the display processing descried above, it is possible for the subject to figure out, from the graph, an overall tendency of temporal changes in vital data on the main screen image, and at the same time check detailed information on a part of the period on the sub screen image. Furthermore, since the amount of the additional information items changes according to a ratio, it is possible for the subject to check necessary information by adjusting the extent of enlargement using the operation of pinch out. The additional information items include additional information items of which the amount of information increases if the range of the specified period expands, such as a measured value, a time and date of measurement, instruction information, and the like, and it is therefore useful to adjust, according to a ratio, the amount of additional information items to be displayed. Since the amount of the additional information items which the sub screen image generating unit 143 displays on the sub screen image corresponds to a ratio, an area of a sub screen image with a lager enlargement by the subject is larger and thus larger amount additional information is displayed than an area of a sub screen image displayed with a small enlargement by the subject. It is to be noted that the sub screen image generating unit 143 may adjust a form of displaying additional information (a character gap, a font type, a font size, etc.) such that all the additional information of the additional information items selected by the display selecting unit 138 is displayed within the region of the sub screen image. This adjustment may be carried out using a maximum font size to the extent allowing all the additional information (character strings forming the additional information) to be displayed within the region of the sub screen image.

[1-2-2. Operation Related to the Instructor Terminal]

The instructor terminal 300 has a function of receiving an input of the priority level information or the instruction information according to an operation performed by the instructor.

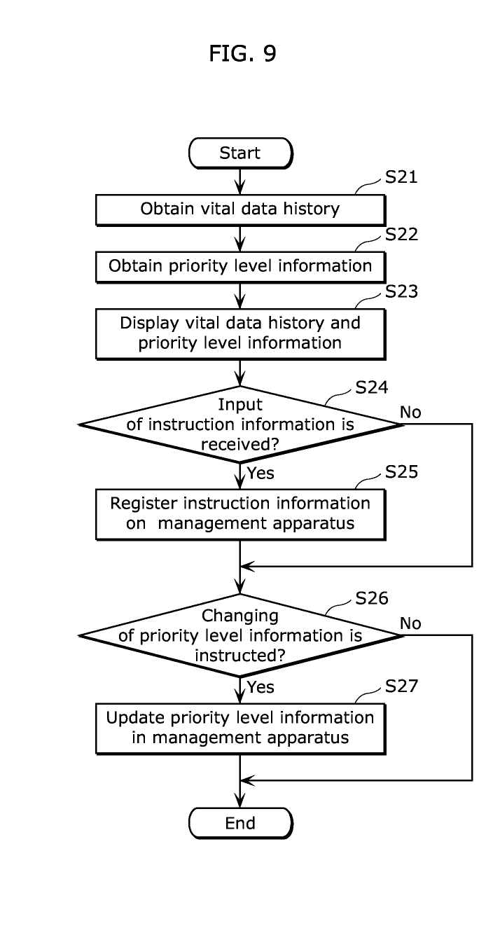

FIG. 9 is a flowchart illustrating an operation of the instructor terminal 300. The following describes an operation of the instructor terminal 300.

First, the instructor terminal 300 receives, trough the input unit 341, an instructor's operation to designate a specific subject, and in response to the operation, obtains, through the vital data obtaining unit 322, vital data history of the specific subject which the instructor intends to view, from the management apparatus 200 (Step S21). In addition, the instructor terminal 300 obtains priority level information concerning the specific subject from the management apparatus 200 (Step S22), and displays the vital data history and the priority level information using the output unit 351 (Step S23). Any form for displaying the vital data history and the priority level information, for example, a tabular form, is applicable, For example, the vital data history may be displayed in a graph form. With this, the instructor is capable of viewing the vital data history and the priority level information of the specific subject displayed on the display 350 of the instructor terminal 300.

When the instructor inputs instruction information, in other words, when the input unit 341 of the instructor terminal 300 receives an instructor's input of instruction information (Step S24), the instruction information is temporarily held in the instruction information holding unit 323, and the instruction information is transmitted to the management apparatus 200 in response to an instruction of the instructor (Step S25). With this, the instruction information is accumulated in the instruction information storage unit 232 of the management apparatus 200.

In addition, when the instructor inputs an instruction for changing the priority level information, in other words, when the input unit 341 of the instructor terminal 300 receives an instructor's instruction for changing the priority level information (and the priority level information after the change) (Step S26), the priority level information after the change is temporarily held in the priority level information holding unit 324. Then, the temporarily held priority level information is transmitted to the management apparatus 200 in response to an instruction from the instructor, thereby updating the priority level information (Step S27). With this, the updated priority level information is stored in the priority level information storage unit 233 of the management apparatus 200. As described above, by updating the priority level information, it is possible for the instructor to establish an order of priority for the various additional information items to be presented to the specific subject

[1-3. Advantageous Effects Etc.]

As described above, according to the present embodiment, the health management system 1 which displays, on a screen of the subject terminal 100, information based on vital data successively obtained from a subject includes: the vital data storage unit 231 which stores vital data history in which the vital data and a time of obtainment are associated with each other; the priority level information storage unit 233 which stores priority level information specifying a priority level for each of a plurality of additional information items; an input and output unit 161 which receives the subject's operation of defining a first region and a second region in the screen; and the display selecting unit 138 which selects an additional information item to be displayed, based on the priority level information and sizes of the first region and the second region of which the operations of defining have been received. The input and output unit 161 displays, on the screen image, the vital data history obtained for a predetermined period, and displays, on a position related to the second region when the operation of defining by the subject is received, additional information of a selected additional information item related to a part of the vital data history corresponding to the first region in the predetermined period

With this, the display selecting unit 138 is capable of changing the amount of additional information items gradually displayed according to a ratio of the defined region, based on the priority level information registered on the management apparatus 200 by the instructor terminal 300, for example. Thus, the subject is capable of checking the additional information, as necessary, on an item selected in relation to the priority level information and the ratio determined by the operation of enlarging the defined region.

### Other Embodiments

As described above, Embodiment 1 has been described as an example of the technique disclosed by the present application. However, the technique according to the present disclosure is not limited to the disclosure in Embodiment 1, and is also applicable to Embodiments to which modifications, replacement, addition, omission, and the like are made. In addition, each of the components described in Embodiment 1 may be combined into a new embodiment.

The following describes a health management system is according to another embodiment.

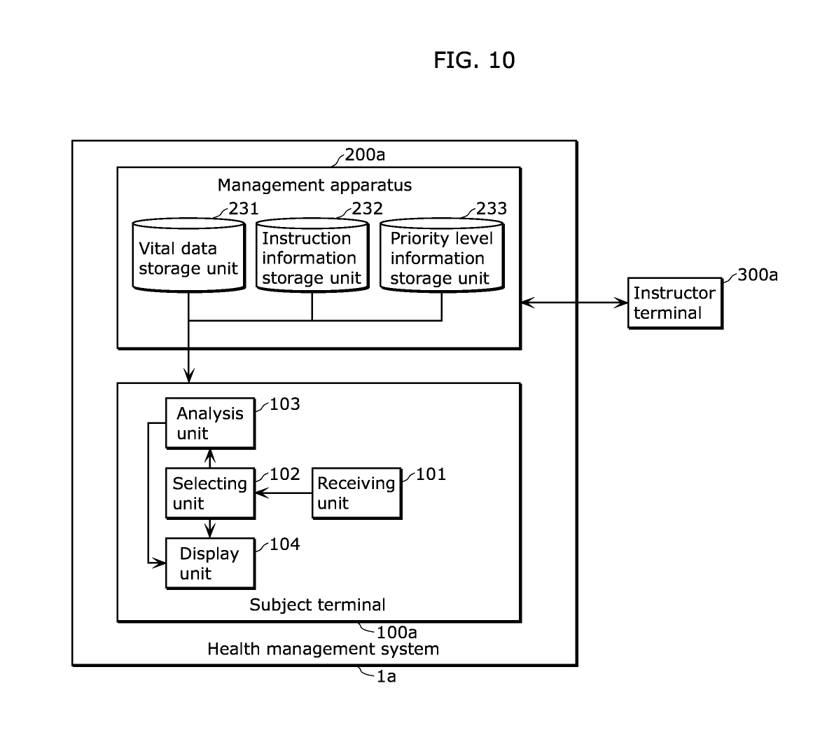

FIG. 10 is a functional block diagram of the health management system is according to another embodiment. The health management system is illustrated in this diagram has a configuration in which a part of the functional configuration of the health management system 1 illustrated in Embodiment 1 is modified, and the instructor terminal is not included. Among the structural elements of the health management system 1a, structural elements same as those in the health management system 1 will be assigned with the same reference numerals of those structural elements described in Embodiment 1, and detailed description for the same structural elements will not be repeated here.

The health management system is includes a management apparatus 200a and a subject terminal 100a. The management apparatus 200a has the same hardware configuration as that of the management apparatus 200 illustrated in Embodiment 1, and functionally includes: the vital data storage unit 231; the instruction information storage unit 232; and the priority level information storage unit 233. The management apparatus 200a is capable of communicating with the subject terminal 100a and the instructor terminal 300a disposed outside the health management system 1a. The instructor terminal 300a has a basically the same configuration as that of the instructor terminal 300 included in the health management system 1. The instructor terminal 300a has a function of downloading vital data history from the management apparatus 200a in response to an instruction of the instructor, uploading instruction information to the instruction information storage unit 232 in the management apparatus 200a, and updating the priority level information stored in the priority level information storage unit 233.

The subject terminal 100a has the same hardware configuration as that of the subject terminal 100 illustrated in Embodiment 1, and functionally includes a receiving unit 101, a selecting unit 102, an analysis unit 103, and a display unit 104. Here, the receiving unit 101 has a function of receiving an operation of defining a first region and a second region in the screen by the subject. For example, the first region is a region shaped in a circle with a diameter that extends between two points which are tapped on a touch panel by the subject, a region shaped in a rectangle with the two points respectively included in opposite corners, or the like, and the second region is, for example, a circle or a rectangle corresponding to the operation of pinch out (enlargement) or pinch in (reduction) of the two tapped points. Examples of the receiving unit 101 include the input and output unit 161, the input detecting unit 131, and the like described in Embodiment 1. The selecting unit 102 has a function of selecting an additional information item to be displayed, based on a size of each of the first region and the second region for which the operation of defining is received by the receiving unit and the priority level information obtained from the priority level information storage unit 233. The selecting unit 102 may perform selection according to a ratio that is a proportion of the size of the second region to the size of the first region, may perform selection according also to the size of the second region (for example, even with the same ratio, the number of additional information items is decreased as the size of the second region is smaller), or may perform selection based on a difference in size between the first region and the second region (difference in a dimension, difference in an area, etc.) (for example, the number of additional information items is increased as the difference is larger). An example of the selecting unit 102 is the display selecting unit 138 according to Embodiment 1. In addition, the analysis unit 103 calculates analysis information on the additional information item selected by the selecting unit 102 based on the vital data history of the part corresponding to the first region. An example of the analysis unit 103 is the analysis information calculating unit 137 according to Embodiment 1. In addition, the display unit 104 has a function of displaying the vital data history obtained for a predetermined period on the screen. The display unit 104, when the receiving unit receives an operation of defining, further has a function of displaying, at a position related to the second region, additional information of the additional information item which is selected by the selecting unit 102 and is related to the vital data history obtained for a part of the predetermined period corresponding to the first region. The position related to the second region is, for example, within the second region or a neighboring region within a predetermined distance from the second region. As to display of the additional information, a display form (a font size, a character gap, etc.) for the additional information may be determined according to the size of the second region, and the additional information may be displayed in the form. Examples of the display unit 104 include the screen image generating unit 141 and the input and output unit 161 according to Embodiment 1.

The following describes a terminal device 100b used by a subject in a health management system according to another embodiment.

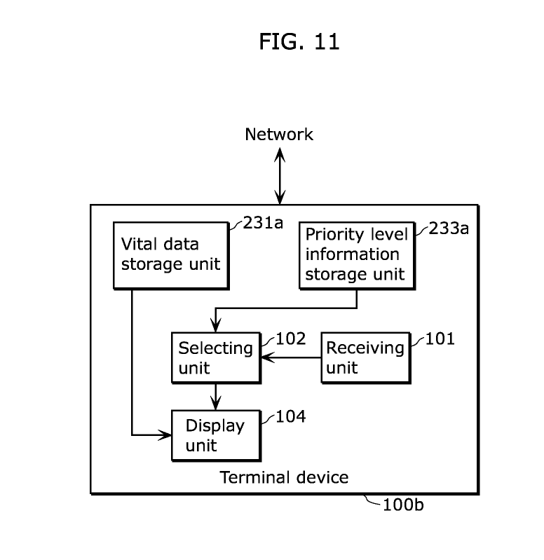

FIG. 11 is a functional block diagram of a terminal device in a health management system according to another embodiment. The terminal device 100b illustrated in the diagram is a terminal device such as a smartphone and a tablet, and includes a memory, a processor, an input device, a display device, a communication I/F, and the like. The terminal device 100b functionally includes: a vital data storage unit 231a; a priority level information storage unit 233a; the receiving unit 101; the selecting unit 102; and the display unit 104. The vital data storage unit 231a is a storage area such as a memory which stores vital data history based on vital data measured for a subject, and may be configured such that the vital data history can be transmitted, via a network, mutually with the vital data storage unit 231 of the management apparatus 200 in the health management system 1. In addition, the priority level information storage unit 233a is a storage area such as a memory which stores priority level information, and may be configured such that the priority level information can be transmitted mutually with the priority level information storage unit 233 of the management apparatus 200. It is to be noted that the receiving unit 101, the selecting unit 102, and the display unit 104 are the same as those included in the subject terminal 100a of the health management system 1a. It is to be noted that the terminal device 100b may include the analysis unit 103 in the case where analysis information is included as an additional information item.

In addition, although the subject terminal 100 is operated by a subject for viewing the vital data history according to Embodiment 1, the subject terminal 100 may be operated for viewing by a person other than the subject (for example, related persons such as a parent of the subject).

In addition, although the measuring device 90 can wirelessly transmit vital data to the subject terminal 100 according to Embodiment 1, the subject terminal 100 may include a measuring device for measuring the vital data (for example, a sphygmomanometer, a pedometer, blood glucose level sensor). In addition, the subject terminal 100 may be electrically connected to the measuring device in a wired manner. Furthermore, the measuring device 90 may transmit and register on the management apparatus 200, without the subject terminal 100, the vital data history in which a time and date of measurement is added to the vital data.

In addition, in the example case of Embodiment 1, the display selecting unit 138 selects additional information (additional information item) to be displayed on the sub screen image by referring to one item of the priority level information for the subject who uses the subject terminal 100. However, the priority level information may be used by selecting, from a plurality of previously prepared priority level information items, a priority level information item for each subject. In other words, a separate priority level information item may be set for each of the subjects who use the health management system, or a common priority level information item may be set for certain subjects.

In addition, the defined region is described as, for example, a circle region in Embodiment 1, however, the defined region is not limited to this. The defined region may be a region shaped in a rectangle with the two points defined by the subject respectively included in opposite corners. In the case of the rectangle region, when a ratio of an original rectangle to an enlarged or reduced rectangle is calculated, it is possible to use, as a ratio, a proportion of the length of either a short side or a long side of the rectangle. Furthermore, although the specified period is detected using perpendiculars which are drawn to a horizontal axis and circumscribe the circle region with a diameter that extends between two points according to Embodiment 1, the specified period may be detected using perpendiculars drawn from the two points.

In addition, although a range of the priority level (lowest priority level) is determined based on a ratio, and an additional information item to be displayed is determined based on the range of the priority level in the example case of Embodiment 1, the additional information may be determined directly from the ratio. If the additional information is determined directly from the ratio, the amount of internal processing is reduced. Furthermore, according to the method of first determining a range of the priority level (lowest priority level) from the ratio, and then determining an additional information item, it is possible to easily correct a relationship between the ratio and the lowest priority level according to a screen size of the subject terminal held by the subject. Furthermore, the ratio table and the priority level information may be integrated. For example, information in which an additional information item to be displayed at a corresponding ratio is associated with the ratio may be used as the priority level information.

In addition, according to Embodiment 1, the ratio detecting unit 133 calculates a ratio (dimensional ratio) that is a proportion of the range (size) of the region which is a target for enlargement or reduction and detected by the region detecting unit 132 to the range (size) of the region resulting from the enlargement or reduction. However, in addition to the dimensional ratio (proportion of the length), an area ratio may be used as the ratio. It is to be noted that a graph to be displayed on the sub screen image (a graph of the vital data history for a specified period) may be displayed in a size to fit within the sub screen independently of the ratio.

In addition, in the example case according to Embodiment 1, the subject defines a region with tapping, and performs the operation of pinch out or pinch in to define a region after enlargement or reduction. However, the tapping, pinch out, and pinch in are merely examples of the operation method, and the defining of the two regions (i.e., defining of the first region and the second region) may be carried out by other operation methods. Although the subject performs the tapping of two points for defining a region shaped in a circle in Embodiment 1, the defining of a region may be carried out by tapping of one point, double tapping, or the like. With the tapping of one point or the like, a region shaped in a circle having a diameter that is previously determined according to the tapping or the like may be defined, by setting the point on which the tapping or the like has been performed as the center of the circle.

In addition, although the vital data history for a specified period is displayed in the sub screen image in a graph form in Embodiment 1, the vital data history is not necessarily displayed in the sub screen image in a graph form, and only additional information may be displayed.

Furthermore, although various processes such as determining the additional information item and calculating analysis information are carried out in the subject terminal 100 in Embodiment 1, all or part of the processes may be shared by the management apparatus 200.

In addition, the order of executing the various processes in the health management system 1 described in Embodiment 1 (for example, the procedures illustrated in FIG. 6 and FIG. 9) is not limited to the order as described in Embodiment 1, and the execution order may be changed unless such changes depart from the scope of the concept of the present disclosure.

In addition, each of the functional structural elements of the devices such as the subject terminal 100, the management apparatus 200, and the instructor terminal 300 described in Embodiment 1 may be installed separately in different devices. Each of the functional structural elements in each of the separate devices operates in cooperation with each other through wired or wireless communication, thereby implementing processes equivalent to those performed by the subject terminal 100, the management apparatus 200, or the instructor terminal 300 described in Embodiment 1.

Furthermore, the functional structural elements (functional blocks) of each of the devices according to the health management system 1 may be made as separate individual chips, or as a single chip to include a part or all thereof, by a semiconductor device such as an integrated circuit (IC) and a large scale integration (LSI). Moreover, ways to achieve integration are not limited to the LSI, and a special circuit or a general purpose processor and so forth can also achieve the integration. Field Programmable Gate Array (FPGA) that can be programmed after manufacturing LSIs or a reconfigurable processor that allows re-configuration of the connection or configuration of circuit cells inside an LSI can be used for the same purpose. Furthermore, in the future, with advancement in semiconductor technology, a brand-new technology may replace LSI. The functional blocks can be integrated using such a technology. The possibility is that the present invention is applied to biotechnology.

Moreover, all or part of each processing (processing procedures or the like shown in FIG. 6 and FIG. 9) performed by the above-mentioned respective devices or the like may be executed by hardware of each device or software. It is to be noted that the execution of the processing by the software is achieved by a processor included in each device or the like executing a control program stored in a memory. In addition, the control program may be recorded on a recording medium and distributed. For instance, a distributed control program is installed into a device or the like, and the device or the like can be caused to execute each processing (processing or the like shown in FIG. 6 and FIG. 9) by causing a processor of the device to execute the control program.

Although only some exemplary embodiments of the present disclosure have been described in detail above, those skilled in the art will readily appreciate that many modifications are possible in the exemplary embodiments without materially departing from the novel teachings and advantages of the present disclosure. Accordingly, all such modifications are intended to be included within the scope of the present disclosure.

### INDUSTRIAL APPLICABILITY

The present disclosure is applicable to a system for displaying information for health management. More specifically, the present disclosure is applicable to the case of sharing information related to the state of health of a patient between a healthcare personnel and the patient.

1. A health management system for displaying, on a screen, information based on vital data successively obtained from a predetermined subject, the health management system comprising:
a vital data storage unit configured to store vital data history in which the vital data is associated with a time at which the vital data is obtained;
a priority level information storage unit configured to store priority level information specifying a priority level for each of a plurality of additional information items;
a receiving unit configured to receive an operation of defining a first region and a second region in the screen;
a selecting unit configured to select an additional information item to be displayed, based on the priority level information and a size of each of the first region and the second region for which the operation of defining is received by the receiving unit; and
a display unit configured to (i) display, on the screen, the vital data history obtained for a predetermined period and (ii) display, on a position related to the second region when the operation of defining is received by the receiving unit, additional information of the additional information item which is selected by the selecting unit and is related to the vital data history obtained for a part of the predetermined period corresponding to the first region.

2. The health management system according to claim 1,
wherein the selecting unit is configured to select the additional information item to be displayed, based on the priority level information according to one of ratios that is a proportion of the size of the second region to the size of the first region, and
the priority level information is information indicating an association between the additional information items and the ratios.

3. The health management system according to claim 2,
wherein, when the operation of defining is received by the receiving unit, the display unit is configured to display the additional information in the second region, and
the selecting unit is configured to select more additional information items as the one of ratios increases.

4. The health management system according to claim 3,
wherein the display unit is configured to determine a display form for the additional information according to the size of the second region, and display the additional information in the determined display form.

5. The health management system according to claim 3,
wherein the selecting unit is configured to select the additional information item to be displayed, based on the priority level information according also to the size of the second region.

6. The health management system according to claim 3,
wherein the display unit is configured to (i) display the vital data history obtained for the predetermined period in a graph form, and (ii) when the operation of defining is received by the receiving unit, enlarge and display in the second region the vital data history in the graph form at the one of ratios that is the proportion.

7. The health management system according to claim 2, further comprising
an analysis unit configured to calculate analysis information of the additional information item selected by the selecting unit, based on the vital data history obtained for a part corresponding to the first region,
wherein the additional information displayed by the display unit includes the analysis information calculated by the analysis unit.

8. The health management system according to claim 2, comprising
a management apparatus which manages the vital data history and a subject terminal which obtains the vital data from the subject, the management apparatus and the subject terminal being capable of communicating with each other,
wherein the subject terminal includes the screen, the receiving unit, and the display unit.

9. The health management system according to claim 8,
wherein the management apparatus is capable of communicating with an instructor terminal used for inputting instruction information for the subject by an instructor who has referred to the vital data history and includes an instruction information storage unit configured to store the instruction information obtained from the instructor terminal, and
the additional information displayed by the display unit includes the instruction information stored in the instruction information storage unit.

10. The health management system according to claim 9,
wherein the priority level information stored in the priority level information storage unit is updatable by the instructor terminal.

11. A terminal device for displaying, on a screen, information based on vital data successively obtained from a subject, the terminal device comprising:
a vital data storage unit configured to store vital data history in which the vital data is associated with a time at which the vital data is obtained;
a priority level information storage unit configured to store priority level information specifying a priority level for each of a plurality of additional information items;
a receiving unit configured to receive an operation of defining a first region and a second region in the screen;
a selecting unit configured to select an additional information item to be displayed, based on the priority level information and a size of each of the first region and the second region for which the operation of defining is received by the receiving unit; and
a display unit configured to (i) display, on the screen, the vital data history obtained for a predetermined period and (ii) display, on a position related to the second region when the operation of defining is received by the receiving unit, additional information of the additional information item which is selected by the selecting unit and is related to the vital data history obtained for a part of the predetermined period corresponding to the first region.

12. A display method for displaying, on a screen of a terminal device, information based on vital data successively obtained from a subject, the display method comprising:
storing vital data history in which the vital data is associated with a time at which the vital data is obtained;
displaying the vital data history obtained for a predetermined period on the screen;
obtaining priority level information specifying a priority level for each of a plurality of additional information items;
receiving an operation of defining a first region and a second region in the screen;
selecting an additional information item to be displayed, based on the priority level information and a size of each of the first region and the second region for which the operation of defining is received; and
displaying, on a position related to the second region, additional information of the additional information item which is selected and is related to the vital data history obtained for a part of the predetermined period corresponding to the first region.

13. A non-transitory computer-readable recording medium for use in a terminal device having a processor, the recording medium having a control program recorded thereon for causing the terminal device to execute display processing of displaying information on a screen, based on vital data successively obtained from a subject, the display processing including:
storing vital data history in which the vital data is associated with a time at which the vital data is obtained;
displaying the vital data history obtained for a predetermined period on the screen;
obtaining priority level information specifying a priority level for each of a plurality of additional information items;
receiving an operation of defining a first region and a second region in the screen;
selecting an additional information item to be displayed, based on the priority level information and a size of each of the first region and the second region for which the operation of defining is received; and
displaying, on a position related to the second region, additional information of the additional information item which is selected and is related to the vital data history obtained for a part of the predetermined period corresponding to the first region.
